(19) United States
(12) Patent Application Publication
(10) Pub. No.: US 2023/0284597 A1
(43) Pub. Date: Sep. 14, 2023

(54) Method and device for judging heat resistance in chickens

(71) Applicant: Huazhong Agricultural University, Wuhan (CN)

(72) Inventors: Yanzhang Gong, Wuhan (CN); Zheya Sheng, Wuhan (CN); Xiaohui Wu, Wuhan (CN)

(21) Appl. No.: 18/199,362

(22) Filed: May 19, 2023

(30) Foreign Application Priority Data
May 19, 2022 (CN) 202210554651.X

(51) Int. Cl. A01K 29/00

(52) U.S. Cl. CPC A01K 29/005

(57) ABSTRACT

A method and device for judging heat resistance of chickens, which can more accurately judge the heat resistance of chickens under “non-lethal” conditions are provided. By treating the chickens with mild heat stress, detecting the blood biochemical index levels of the chickens before and after heat stress, and substituting them into the heat resistance judgment model, in such a manner that the heat resistance of the chickens is judged.

### CROSS REFERENCE OF RELATED APPLICATION

The present application claims priority under 35 U.S.C. 119(a-d) to CN 202210554651.X, filed May 19, 2022.

### BACKGROUND OF THE PRESENT INVENTION

### Field of Invention

The present invention relates to breeding of heat-resistant chickens, and more particularly to a method and device for evaluating heat-resistance chickens.

### Description of Related Arts

With the rapid development of China's economy in the past few decades and the improvement of people's living standards, the demand for meat, eggs, milk and other livestock and poultry products has also continued to increase. Among livestock and poultry products, poultry meat and poultry eggs are cheap, nutritious and delicious, and can be widely accepted by groups with different cultural backgrounds and religious beliefs, and poultry breeding industry plays an important role in promoting agricultural and rural economic development in China. Meanwhile, poultry breeding industry of China ranks front row in the world in terms of breeding volume, egg production and consumption, and plays an important leading role in the development of Chinese animal husbandry industry. Therefore, it is of great significance to ensure the healthy and stable development of poultry breeding industry. However, due to the continuous warming of the global climate, among the five major climate types in China, except for the plateau and mountainous climate in the western region, other climate types have the problem of high temperature in summer, and the modern poultry industry is highly intensified and most poultry have experienced various factors such as high-intensity genetic selection lead to heat stress, which is an unavoidable problem in Chinese poultry farming industry in summer. Affected by heat stress, the growth of poultry is inhibited, the production performance is reduced, the immune function is weakened, and the susceptibility to diseases is weakened. In severe cases, it will directly lead to death, causing significant economic losses to the poultry farming industry.

At present, equipment such as wet curtains, mist lines and fans are often used in production to relieve heat stress through physical methods, but there is a large cost of manpower and material resources. There is also a method of adding antipyretic drugs to feed and drinking water, but It will cause certain toxic and side effects to chickens, and these methods cannot completely eliminate the impact of heat stress. Only by improving the heat resistance of the chicken itself and breeding heat-resistant chickens can the heat stress problem be fundamentally solved. However, the heat stress survival time at 40° C. (HSST40), which is recognized as the most accurate indicator for evaluating the heat tolerance of chickens, is a lethal indicator and cannot be directly used in actual production and research. Therefore how to more accurately judge the heat-resistant performance of chicken under “non-lethal” condition is exactly the key point of carrying out heat-resistant chicken breeding.

### SUMMARY OF THE PRESENT INVENTION

Based on the above background, the present invention provides a method and device for judging heat resistance of chickens, which can more accurately judge the heat resistance of chickens under “non-lethal” conditions. By treating the chickens with mild heat stress, detecting the blood biochemical index levels of the chickens before and after heat stress, and substituting into the heat resistance judgment model, so as to judge the heat resistance of the chickens.

Technical solutions of the present invention are as follows.

A device for judging heat-resistance chickens, comprises: a heat resistance discrimination module and a display module, wherein a discriminant function is stored in the heat resistance discrimination module: y=−1.20×10⁻¹×x₁+7.80×10⁻¹×x₂−6.37×10⁻²×x₃+5.86×10⁻⁴×x₄+9.43×10⁻³×x₅−2.47×10⁻¹×x₆−1.50;

wherein the heat resistance discrimination module is configured to discriminate the heat resistance of chickens according to the blood biochemical index and blood gas index data of chickens before and after mild heat stress treatment;

wherein y represents the heat tolerance of the chicken, x₁ is a first TCHO concentration of the chicken before mild heat stress, x₂ is a second TCHO concentration of the chicken after mild heat stress, x₃ is a Hct level of the chicken after mild heat stress, x₄ is a concentration of CK after mild heat stress in the chicken, x₅ is a difference in the concentration of AST before and after the mild heat stress in the chicken, x₆ is a difference in the concentration of ALB before and after the mild heat stress in the chicken, when an output of y is less than 0, the display module shows that the chicken is heat-labile, and when the output of y is greater than or equal to 0, the display module shows that the chicken is heat-resistant;

wherein the mild heat stress treatment is to transfer the non-heat-stressed chickens to an environment at 32±1° C. and a relative humidity of 60% to 70% for 6 hours.

A method for judging heat-resistance chickens, comprises steps of:
Step (1): collecting blood of chickens which are not subjected to heat stress to detect concentrations of TCHO, AST and ALB;
Step (2): treating the chickens in step (1) with mild heat stress: transfering the chickens which are not subjected to heat stress to an environment at 32±1° C. and a relative humidity of 60% to 70%, and keeping for 6 h;
Step (3): collecting the blood of the chickens treated in step (2) to obtain TCHO concentration, Hct level, CK concentration, AST concentration, and ALB concentration; and
Step (4): obtaining based on the parameters of step (1) and step (2): x₁ is the TCHO concentration of the chicken before mild heat stress, x₂ is the TCHO concentration of the chicken after mild heat stress, x₃ is the Hct of the chicken after mild heat stress level, x₄ is the CK concentration after mild heat stress in chickens, x₅ is the difference in AST concentration before and after mild heat stress in chickens, x₆ is the difference in ALB concentration before and after mild heat stress in chickens, and the above parameters are brought into a discriminant function: y=−1.20×10⁻¹×x₁+7.80×10⁻¹×x₂−6.37×10⁻²×x₃+5.86×10⁻⁴×x₄+9.43×10⁻³×x₅−2.47×10⁻¹×x₆−1.50; wherein y represents the heat resistance of the chicken, when the y output is less than 0, the chicken is not heat resistant, when the y output is greater than or equal to 0, the chicken is heat resistant.

The discriminant function of the present invention is obtained through the following steps:
1. Designing temperature-controlled cabin for heat stress treatment
The temperature control cabin is 6 m long, 5 m wide, and 3 m high. The overall space is sealed, and polyurethane materials are used for heat insulation. Install heating equipment and humidification equipment on both sides of the long axis, and use a high-precision thermostat to control the space temperature.
2. A chicken heat tolerance judgment method, comprising:
(1) subjecting the sample chickens to mild heat stress treatment first, wherein the blood biochemical indicators and blood gas indicators of the chickens were detected before and after the heat stress, and then heat shock treatment at 40° C. was performed to group the sample chickens according to their heat resistance;
(2) establishing a chicken heat tolerance judgment model: According to the HSST40 of the chicken, the sample chickens are divided into heat-resistant and heat-intolerant, and the various indicators and their change levels of chickens with different heat tolerance before and after mild heat stress Carry out independent sample T test, use stepwise linear regression to screen the indicators with significant differences, and use the screened indicators to establish the Fisher discriminant function of heat resistance;
(3) collecting the data of blood biochemical indicators and blood gas indicators before and after the mild heat stress treatment of the chickens to be judged, and substitute them into the heat resistance judgment model of the chickens to obtain the heat resistance results of the chickens.

The mild heat stress treatment described is specifically:
Raising the chickens in a thermoneutral environment for more than two weeks to ensure that the chickens are not affected by heat stress, and then transfering the chickens to a temperature-controlled cabin with an ambient temperature of 32±1° C. and a relative humidity of 60%-70% for heat stress treatment, collecting 1 mL of subwing venous blood from chickens before and after heat stress treatment;

Described 40° C. heat shock treatment specifically comprising:
Feeding the chickens in a thermoneutral environment for more than two weeks to eliminate the effects of the mild heat stress treatment on the chickens, and then transfering the chickens to a room with an ambient temperature of 40±1° C. and a relative humidity of 45%-55%; carrying out heat shock treatment in the temperature control cabin, and recording the HSST40 of each chicken manually;

The screened blood biochemical indicators and blood gas indicators are as follows:
Serum total cholesterol (TCHO) before mild heat stress treatment, serum total cholesterol (TCHO) after mild heat stress treatment, albumin (ALB), aspartate aminotransferase (AST), creatine kinase (CK) and whole blood red blood cells Specific volume (Hct) has a total of 6 indicators;
Set the sample data of chickens with HSST40<120 min after heat stress treatment at 32±1° C. as the heat-labile group, and set the sample data of chickens with HSST40 [symbol] 120 min after heat stress at 32±1° C. as the heat-resistant group to establish Fisher Discriminant function, the sample size of each group is ni, and the total sample size is n.

First, calculate the mean value of each group according to

$${\overline{X}}_{i} = \frac{1}{{n}_{i}} {\sum }_{j = 1}^{{n}_{i}} {x}_{( j )}^{i} ,$$

wherein i is a different group, and i→1 is The non-heat-resistant group, i→2 is the heat-resistant group, and j is the symbol of the index.

$${\overline{X}}_{1} = \frac{1}{{n}_{1}} {\sum }_{j = 1}^{{n}_{1}} {x}_{( j )}^{1} = {[ {x}_{( preTCHO )}^{1} , {x}_{( postTCHO )}^{1} , {x}_{( postHct )}^{1} , {x}_{( postCK )}^{1} , {x}_{( difAST )}^{1} , {x}_{( difALB )}^{1} ]}^{T} ; \\ {\overline{X}}_{2} = \frac{1}{{n}_{2}} {\sum }_{j = 1}^{{n}_{2}} {x}_{( j )}^{2} = {[ {x}_{( preTCHO )}^{2} , {x}_{( postTCHO )}^{2} , {x}_{( postHct )}^{2} , {x}_{( postCK )}^{2} , {x}_{( difAST )}^{2} , {x}_{( difALB )}^{2} ]}^{T} ;$$

According to average means of each group, calculating an overall mean;

$$\overline{X} = \frac{1}{{n}_{1} + {n}_{2}} ( {n}_{1} {\overline{X}}_{1} + {n}_{2} {\overline{x}}_{2} )$$

After obtaining the overall mean, calculate the covariance matrix Sᵢ of each group and the covariance matrix Sₚ within the joint group, the SSCP matrix W within the group and the SSCP matrix B between the groups, wherein X₍ⱼ₎ⁱ is the jth sample of the i-th group:

$${S}_{1} = \frac{1}{{n}_{1} - 1} {\sum }_{j = 1}^{{n}_{1}} ( {X}_{( j )}^{1} - \overline{{X}_{1}} ) {( {X}_{( j )}^{1} - \overline{{X}_{1}} )}^{T} , \\ {S}_{2} = \frac{1}{{n}_{2} - 1} {\sum }_{j = 1}^{{n}_{2}} ( {X}_{( j )}^{2} - \overline{{X}_{2}} ) {( {X}_{( j )}^{2} - \overline{{X}_{2}} )}^{T} ,$$

$${S}_{p} = \frac{1}{{n}_{1} + {n}_{2} - 2} [ ( {n}_{1} - 1 ) {S}_{1} + ( {n}_{2} - 1 ) {S}_{2} ] , \\ W = {\sum }_{i = 1}^{2} {\sum }_{j = 1}^{{n}_{i}} ( {X}_{( j )}^{i} - {\overline{X}}_{i} ) {( {X}_{( j )}^{i} - {\overline{X}}_{i} )}^{T} , \\ B = {\sum }_{i = 1}^{2} {n}_{i} ( {\overline{X}}_{i} - \overline{X} ) {( {\overline{X}}_{i} - \overline{X} )}^{T} ,$$

The “T” symbol is to take the transposed matrix. After obtaining W and B to calculate the characteristic root k of the discriminant function, the number of roots t is min (p, g−1), which is the number of discriminant functions, and p is related to heat resistance The number of indicators with strong sex, p=6, g=2. Then calculate the characteristic root according to (W″−1 ″B−λ I) E=0, I and E are unit matrix; finally get k and calculate the coefficient a“t” of each index in the discriminant function:

According to (W⁻¹ B−λₜ I)aₜ=0, (aₜ^{T} Sₚ aₜ)=1, and cₜ=−aₜ^{T}X̅ to obtain the discriminant function: y=−1.20×10⁻¹×x₁+7.80×10⁻¹×x₂−6.37×10⁻²×x₃+5.86×10⁻⁴×x₄+9.43×10⁻³×x₅−2.47×10⁻¹×x₆−1.50;

wherein y represents the heat tolerance of the chicken, x₁ is the TCHO concentration of the chicken before mild heat stress, x₂ is the TCHO concentration of the chicken after mild heat stress, x₃ is the Hct level of the chicken after mild heat stress, x₄ is the concentration of CK in chickens after mild heat stress, x₅ is the difference in AST concentration in chickens before and after mild heat stress, and x6 is the difference in ALB concentration in chickens before and after mild heat stress. When the y output is less than 0, it is judged that the chicken is heat-labile (HSST40<120 min), when the y Output is greater than or equal to 0, it is judged that the chicken is heat-resistant (HSST40 [symbol] 120 min).

The beneficial effects that the present invention has are as follows.

The invention can be used to judge the heat resistance of different breeds of laying hens;

The invention can more accurately judge the heat resistance of chickens under the “non-lethal” condition.

These and other objectives, features, and advantages of the present invention will become apparent from the following detailed description, the accompanying drawings, and the appended claims.

### BRIEF DESCRIPTION OF THE DRAWINGS

FIG. 1 is a flow chart of the method of the present invention.

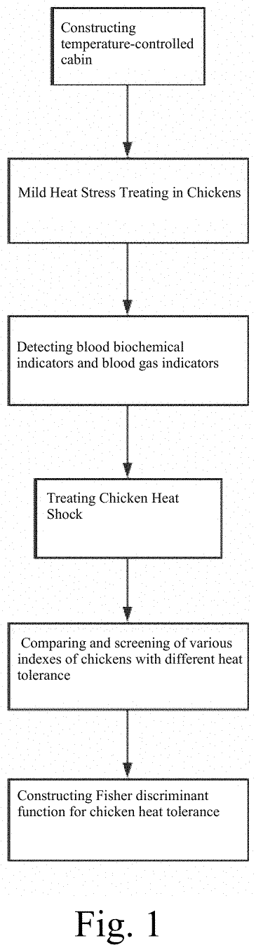

FIG. 2 is a structural schematic diagram of a temperature control cabin of the present invention.

FIG. 3 is HSST40 situation of Hailan brown layer hens and Xinhua layer hens according to a preferred embodiment of the present invention.

FIG. 4 is an index ROC curve inspection result after screening according to the preferred embodiment of the present invention

### DETAILED DESCRIPTION OF THE PREFERRED EMBODIMENT

Preferred embodiment of the present invention are described below in conjunction with the accompanying drawings:

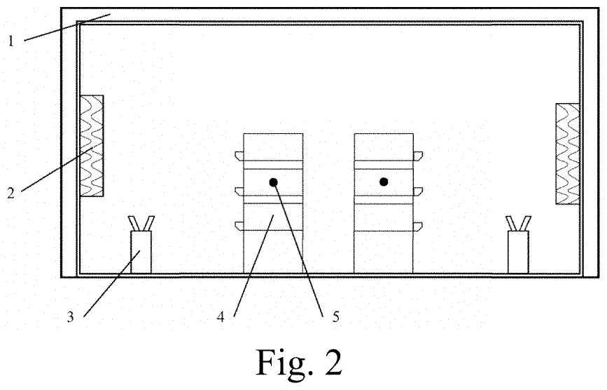

The overall structure of the temperature control cabin of the present invention is symmetrical, as shown in FIG. 2, comprising an outer polyurethane insulation structure 1, an internal heating device 2, a humidifying device 3, a chicken cage 4 and a temperature-sensitive probe 5; a heating device with a power of 8 kW 2 is installed in the middle of the walls on both sides of the long axis, and blows air to the ground at a speed of 2.6 m/s; the humidification device 3 is placed between the heating device and the chicken coop, and the start-up of the humidification device 3 is manually controlled according to the data of the temperature and humidity meter. Stop; the chicken cage 4 is a three-layer structure, located on both sides of the middle of the room; the temperature-sensitive probe 5 is located in the middle layer of the chicken cage 4, detects the real-time temperature and controls the start and stop of the heating device 2, thereby maintaining the temperature of the temperature control cabin.

### EMBODIMENT

1) 70 Xinhua laying hens and 75 Hailan brown laying hens are used. The chickens are at the peak of egg production. Under normal circumstances, the chickens are kept in a thermoneutral environment for more than two weeks to ensure that the chickens are not exposed to heat the effects of stress;
2) Transfer the chickens to a temperature-controlled cabin that has been maintained at 32±1° C. and a relative humidity of 60%-70% for 6 hours of mild heat stress treatment, and before and after the heat stress treatment, the chickens were injected from the subwing vein. 1 mL of blood was collected for detection of blood biochemical indicators and blood gas indicators (Table. 1);

| Biochemical indicators | Blood gas indicators |
|---|---|
| Total Cholesterol (TCHO) | Total Carbon Dioxide (TCO₂) |
| Triglycerides (TG) | Partial pressure of carbon dioxide (PCO₂) |
| Total protein (TP) | Bicarbonate ion (HCO₃⁻) |
| Albumin (ALB) | Sodium ion (Na⁺) |
| Creatine Kinase (CK) | Chloride Ion (Cl⁻) |
| Lactate dehydrogenase (LDH) | Potassium ion (K⁺) |
| Cereal grass transaminase | Anion gap (AnGap) |
| (AST) | pH |
| Superoxide dismutase (SOD) | Base Excess (BEecf) |
| Cortisol (Cortisol) | Hematocrit (Hct) |
| Tetraiodothyronine (T4) | Glucose (Glu) |

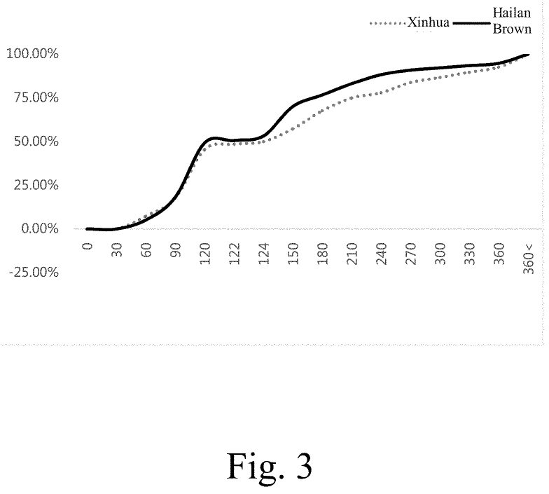

4) Transfer the chickens after mild heat stress to a heat-neutral environment and raise them for more than two weeks. Transfer the chickens to a temperature-controlled cabin that has been kept at 40±1° C. and a relative humidity of 45% to 55% for heat shock treatment, and manually observe and record the HSST40 (heat stress survival time) of each chicken;
5) The HSST40 conditions of Hailan brown layer and Xinhua layer are shown in FIG. 3. The half heat shock death time of Hailan brown layer is 122 minutes, and the half heat shock death time of Xinhua layer is 124 minutes, so With 120 min as the boundary, HSST40<120 min is heat-labile, HSST40 [symbol] 120 min is heat-resistant, and the chickens are classified according to this;
6) Perform independent sample T-test on the heat-resistant chickens and non-heat-resistant chickens under mild heat stress before heat (pre), after heat (post) and the difference before and after heat (dif), and get Table 2, the indicators shown in Table 2 are indicators with significant differences detected between heat-resistant and heat-labile chickens before heat (pre) and after heat (post), and before heat (pre) and after heat (post) post) index difference with significant difference (P<0.05);

TABLE 2
Significant difference indexes of chickens with different heat tolerance

| Heat | Before | Meam ± standard | After | Meam ± standard | | Meam ± standard |
|---|---|---|---|---|---|---|
| resistance | heat | deviation | heat | deviation | Difference | deviation |
| Heat- | pre | 2.38 ± 0.67 | post | 2.21 ± 0.73 | dif | 16.88 ± 31.21 |
| labile | TCHO | | TCHO | | AST | |
| Heat | | 2.79 ± 0.89 | | 2.55 ± 0.87 | | 30.01 ± 40.06 |
| resistance | | | | | | |
| Heat- | pre | 13.11 ± 9.03 | post | 23.83 ± 2.57 | dif | −0.69 ± 1.11 |
| labile | TG | | TCO₂ | | ALB | |
| Heat | | 17.32 ± 11.25 | | 24.65 ± 2.15 | | −1.07 ± 1.06 |
| resistance | | | | | | |
| Heat- | pre | 46.33 ± 5.00 | post | 31.38 ± 9.30 | dif | 0.024 ± 0.084 |
| labile | TP | | Hct | | pH | |
| Heat | | 48.21 ± 5.19 | | 26.34 ± 6.58 | | 0.054 ± 0.087 |
| resistance | | | | | | |
| Heat- | pre | 15.58 ± 1.15 | post | 10.65 ± 3.16 | | |
| labile | ALB | | Hb | | | |
| Heat | | 16.07 ± 1.33 | | 8.94 ± 2.23 | | |
| resistance | | | | | | |
| Heat- | pre | 234.76 ± 23.17 | post | 1501.02 ± 290.81 | | |
| labile | Glu | | SOD | | | |
| Heat | | 243.88 ± 23.39 | | 1622.49 ± 352.38 | | |
| resistance | | | | | | |
| Heat- | pre | 33.21 ± 6.96 | post | 2072.84 ± 840.42 | | |
| labile | PCO₂ | | CK | | | |
| Heat | | 36.03 ± 7.73 | | 2466.6 ± 840.23 | | |
| resistance | | | | | | |

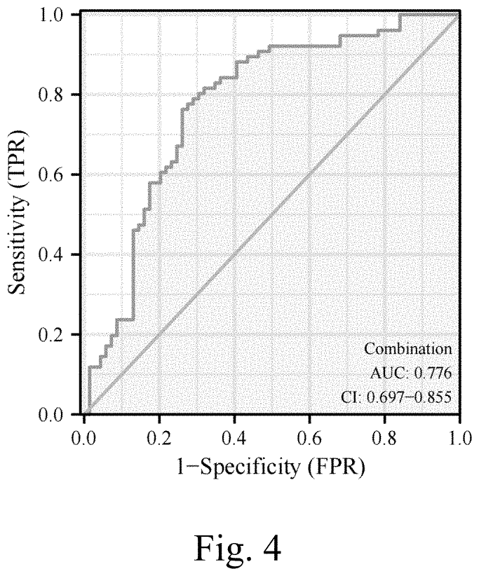

7) Perform a stepwise regression analysis on the indicators with significant differences in the pre-heating, post-heating and difference shown in Table 2 and heat resistance in turn. Heat resistance is the classification result of chickens. The results are shown in Table 3, Table 4, As shown in Table 5, Table 6, Table 7 and Table 8, the six indicators of preTCHO, postTCHO, postHct, postCK, difAST and difALB were finally screened, and the six indicators were tested together by the ROC curve, and the results are shown in FIG. 4, with an AUC of 0.776 and moderate predictive accuracy;

TABLE 3
Screening results of significant difference indicators before heat

| | | | | Standard | | | |
|---|---|---|---|---|---|---|---|
| | | | Adjusted | Estimated | R₂ | F | Signifi- |
| Model | R | R² | R² | Error | change | change | cance P |
| 1 | 0.249 | 0.062 | 0.055 | 0.48709 | 0.062 | 9.432 | 0.003 |

1 predictor variable: (constant), preTCHO

TABLE 4
Results of excluded variables of significant difference indicators before heat

| | | | | | | Collinearity |
|---|---|---|---|---|---|---|
| | Excluded | Inout | | | Partial | Statistical |
| Model | Variables | Beta | t | Significance | Correlation | Tolerance |
| 1 | preTG | 0.064 | 0.58 | 0.563 | 0.049 | 0.548 |
| | preTP | 0.059 | 0.598 | 0.551 | 0.05 | 0.671 |
| | preALB | 0.092 | 0.987 | 0.325 | 0.083 | 0.754 |
| | preGluWB | 0.157 | 1.929 | 0.056 | 0.16 | 0.973 |
| | prePCO2 | 0.134 | 1.61 | 0.11 | 0.134 | 0.934 |

TABLE 5
Screening results of significant difference indicators after heating

| | | | | Standard | | | Signifi- |
|---|---|---|---|---|---|---|---|
| | | | Adjusted | Estimated | R₂ | F | cance |
| Model | R | R² | R² | Error | change | change | P |
| 1 | 0.302 | 0.091 | 0.085 | 0.47935 | 0.091 | 14.394 | 0.000 |
| 2 | 0.365 | 0.133 | 0.121 | 0.46989 | 0.042 | 6.818 | 0.010 |
| 3 | 0.422 | 0.178 | 0.160 | 0.45926 | 0.045 | 7.645 | 0.006 |

1 Predictor variable: (constant), postHct
2 Predictor variable: (constant), postHct, postCK
3 Predictor variable: (constant), postHct, postCK, postTCHO

TABLE 6
Excluded variable results of significant difference indicators
after heat treatment

| | | | | | | Collinearity |
|---|---|---|---|---|---|---|
| | Excluded | Input | | Signifi- | Partial | Statistical |
| Model | Variables | Beta | t | cance | Correlation | Tolerance |
| 1 | postTCHO | 0.195 | 2.488 | 0.014 | 0.204 | 0.998 |
| | postCK | 0.205 | 2.611 | 0.01 | 0.214 | 0.993 |
| | postTCO2 | 0.158 | 1.998 | 0.048 | 0.165 | 0.998 |
| | postHb | 6.684 | 0.892 | 0.374 | 0.075 | 0 |
| | postSOD | 0.149 | 1.867 | 0.064 | 0.155 | 0.984 |
| 2 | postTCHO | 0.212 | 2.765 | 0.006 | 0.227 | 0.992 |
| | postTCO2 | 0.142 | 1.818 | 0.071 | 0.151 | 0.991 |
| | postHb | 8.714 | 1.183 | 0.239 | 0.099 | 0 |
| | postSOD | 0.136 | 1.735 | 0.085 | 0.145 | 0.98 |
| 3 | postTCO2 | 0.121 | 1.576 | 0.117 | 0.132 | 0.98 |
| | postHb | 12.916 | 1.773 | 0.078 | 0.148 | 0 |
| | postSOD | 0.132 | 1.72 | 0.088 | 0.144 | 0.979 |

TABLE 7
Screening results of significant difference indicators before and
after heating

| | | | | Standard | | | Signifi- |
|---|---|---|---|---|---|---|---|
| | | | Adjusted | Estimated | R₂ | F | cance |
| Model | R | R² | R² | Error | change | change | P |
| 1 | 0.180 | 0.032 | 0.026 | 0.49471 | 0.032 | 4.773 | 0.031 |
| 2 | 0.269 | 0.072 | 0.059 | 0.4861 | 0.04 | 6.109 | 0.015 |

1 Predictor variable: (constant) difAST
2 Predictor variable: (constant), difAST, difALB

TABLE 8
The results of the significant difference index excluded variables

| | Excluded | | | | | Collinearity |
|---|---|---|---|---|---|---|
| | Variables | Input | | | Partial | Statistical |
| Model | Input | Beta | t | Significance | Correlation | Tolerance |
| 1 | difALB | −0.202 | −2.472 | 0.015 | −0.203 | 0.982 |
| | difpH | 0.154 | 1.877 | 0.063 | 0.156 | 0.987 |
| 2 | difpH | 0.099 | 1.154 | 0.25 | 0.097 | 0.883 |

Use the remaining samples of preTCHO, postTCHO, postHct, postCK, difAST and difALB after screening and regrouping, and set the sample data of chickens with HSST40<120 min after heat stress treatment at 32±1° C. as intolerant For the heat group, set the sample data of chickens with HSST40≥120 min after heat stress at 32±1° C. as the heat-resistant group, and establish the Fisher discriminant function. The sample size of each group is ni, and the total sample size is n.

First, calculate the mean value of each group according to

$${\overline{X}}_{i} = \frac{1}{{n}_{i}} {\sum }_{j = 1}^{{n}_{i}} {x}_{( j )}^{i} ,$$

wherein i is a different group, and i→1 is The non-heat-resistant group, i→2 is the heat-resistant group, and j is a symbol of the index.

$${\overline{X}}_{1} = \frac{1}{{n}_{1}} {\sum }_{j = 1}^{{n}_{1}} {x}_{( j )}^{1} = {[ {x}_{( preTCHO )}^{1} , {x}_{( postTCHO )}^{1} , {x}_{( postHct )}^{1} , {x}_{( postCK )}^{1} , {x}_{( difAST )}^{1} , {x}_{( difALB )}^{1} ]}^{T} ; \\ {\overline{X}}_{2} = \frac{1}{{n}_{2}} {\sum }_{j = 1}^{{n}_{2}} {x}_{( j )}^{2} = {[ {x}_{( preTCHO )}^{2} , {x}_{( postTCHO )}^{2} , {x}_{( postHct )}^{2} , {x}_{( postCK )}^{2} , {x}_{( difAST )}^{2} , {x}_{( difALB )}^{2} ]}^{T} ;$$

Then calculate the overall mean based on the mean of each group:

$$\overline{X} = \frac{1}{{n}_{1} + {n}_{2}} ( {n}_{1} {\overline{X}}_{1} + {n}_{2} {\overline{x}}_{2} )$$

After obtaining the overall mean, calculate the covariance matrix Sᵢ of each group and the covariance matrix Sₚ within the joint group, the SSCP matrix W within the group and the SSCP matrix B between the groups, wherein X₍ⱼ₎ⁱ is the jth sample of the ith group:

$${S}_{1} = \frac{1}{{n}_{1} - 1} {\sum }_{j = 1}^{{n}_{1}} ( {X}_{( j )}^{1} - \overline{{X}_{1}} ) {( {X}_{( j )}^{1} - \overline{{X}_{1}} )}^{T} , \\ {S}_{2} = \frac{1}{{n}_{2} - 1} {\sum }_{j = 1}^{{n}_{2}} ( {X}_{( j )}^{2} - \overline{{X}_{2}} ) {( {X}_{( j )}^{2} - \overline{{X}_{2}} )}^{T} , \\ {S}_{p} = \frac{1}{{n}_{1} + {n}_{2} - 2} [ ( {n}_{1} - 1 ) {S}_{1} + ( {n}_{2} - 1 ) {S}_{2} ] , \\ W = {\sum }_{i = 1}^{2} {\sum }_{j = 1}^{n} ( {X}_{( j )}^{i} - {\overline{X}}_{i} ) {( {X}_{( j )}^{i} - {\overline{X}}_{i} )}^{T} , \\ B = {\sum }_{i = 1}^{2} {n}_{i} ( {\overline{X}}_{i} - \overline{X} ) {( {\overline{X}}_{i} - \overline{X} )}^{T} ,$$

The “T” symbol is to take the transposed matrix. After obtaining W and B to calculate the characteristic root λ of the discriminant function, the number of roots t is min (p, g−1), which is the number of discriminant functions, and p is related to heat resistance The number of indicators with strong sex, p=6, g=2. Then calculate the characteristic root according to (W^{·1}B−λ I) E=, I and E are unit matrix: finally get λ and calculate the coefficient at of each index in the discriminant function: according to (W⁻¹ B−λₜ I)aₜ=0, (aₜ^{T} Sₚ aₜ)=1 and cₜ=−aₜ^{T}X̅. The obtained discriminant function is used to construct the Fisher discriminant function model of chicken heat resistance through Fisher discriminant analysis, and its formula is:

y=−1.20×10⁻¹×x₁+7.80×10⁻¹×x₂−6.37×10⁻²×x₃+5.86×10⁻⁴×x₄+9.43×10⁻³×x₅−2.47×10⁻¹×x₆−1.50;

wherein y represents the heat tolerance of the chicken, x₁ is the TCHO concentration of the chicken before mild heat stress, x₂ is the TCHO concentration of the chicken after mild heat stress, x₃ is the Hct level of the chicken after mild heat stress, x₄ is the CK concentration of the chicken after mild heat stress, x₅ is the AST of the chicken before and after mild heat stress difference in concentration change, x₆ is difference in ALB concentration before and after mild heat stress in chickens. When y<0, it is judged that the chicken is heat-labile, and when y≥0, it is judged that the chicken is heat-resistant.
8) Use the established Fisher discriminant function model of heat resistance of chickens to distinguish the heat resistance of chickens, and use the function to distinguish the heat-resistant chicken group and non-heat-resistant chicken group in Xinhua chicken and Hailan chicken according to the indicators in step (6). The heat-resistant chicken group is checked with the actual heat-resistant chicken group and heat-resistant chicken group screened in step (4), and then the discrimination accuracy rate is calculated. The judgment results of some chickens are shown in Table 9, and the overall judgment of the model is accurate The rate was 75.2%, among which 21 Xinhua laying hens were misjudged, with an accuracy rate of 70.0%, and Hailan brown layer hens were misjudged 15, with an accuracy rate of 80.0%. The model can accurately judge the heat resistance of chickens.

TABLE 9
Judgment results of Fisher's discriminant function for heat tolerance of some chickens

| | | Actual | | | | | | | | Predicted |
|---|---|---|---|---|---|---|---|---|---|---|
| Chicken | | heat | | | | | | | | heat |
| number | HSST40 | resistance | preTCHO | postCK | postTCHO | postHct | difAST | difALB | y | resistance |
| Xinhua 1 | 56 | heat-labile | 2.55 | 1368 | 2.43 | 27 | 50 | −0.5 | −0.234 | heat-labile |
| Xinhua 2 | 75 | heat-labile | 2.17 | 2189 | 2.56 | 40 | 27.95 | 1.52 | −1.141 | heat-labile |
| Xinhua 3 | 91 | heat-labile | 2.51 | 2084.9 | 2.32 | 27 | 20.5 | −0.93 | −0.067 | heat-labile |
| Xinhua 4 | 99 | heat-labile | 2.96 | 1705 | 2.94 | 26 | 12 | −1.3 | 0.215 | Heat- |
| | | | | | | | | | | resistance |
| Xinhua 5 | 106 | heat-labile | 2.85 | 1781 | 3.53 | 46 | 9 | −0.1 | −0.866 | heat-labile |
| Xinhua 6 | 144 | Heat- | 3.01 | 2662 | 2.35 | 20 | 20 | −1.6 | 0.842 | Heat- |
| | | resistance | | | | | | | | resistance |
| Xinhua 7 | 160 | Heat- | 3.34 | 619 | 2.88 | 27 | 41 | −2.2 | −0.082 | heat-labile |
| | | resistance | | | | | | | | |
| Xinhua 8 | 200 | resistance | 2.11 | 2649 | 1.68 | 18 | 37 | −2 | 0.806 | Heat- |
| | | | | | | | | | | resistance |
| Xinhua 9 | 245 | resistance | 2.27 | 2541 | 1.43 | 19 | 92 | −2 | 0.983 | Heat- |
| | | | | | | | | | | resistance |
| Xinhua | 342 | resistance | 4.61 | 2535 | 3.66 | 20 | 26 | −0.6 | 1.406 | Heat- |
| 10 | | | | | | | | | | resistance |
| Hailan | 60 | heat-labile | 1.88 | 2279 | 1.63 | 62 | 25 | −0.3 | −2.758 | heat-labile |
| Brown 1 | | | | | | | | | | |
| Hailan | 75 | heat-labile | 1.29 | 1751 | 1.36 | 38 | −32 | −1.2 | −1.994 | heat-labile |
| Brown 2 | | | | | | | | | | |
| Hailan | 94 | heat-labile | 1.61 | 966 | 1.69 | 39 | 9 | −0.6 | −2.060 | heat-labile |
| Brown 3 | 104 | heat-labile | 2.58 | 2932 | 2.15 | 20 | 14 | −1.1 | 0.715 | Heat- |
| Hailan | | | | | | | | | | resistance |
| Brown 4 | | | | | | | | | | |
| Hailan | 116 | heat-labile | 1.84 | 952 | 1.99 | 29.65 | 66 | −0.1 | −0.852 | heat-labile |
| Brown 5 | 1 | | | | | | | | | |
| Hailan | 37 | Heat | 1.59 | 1704 | 1.63 | 30 | −7 | −0.1 | −1.373 | heat-labile |
| Brown 6 | | resistance | | | | | | | | |
| Hailan | 161 | Heat | 2.18 | 3088 | 2.41 | 24.85 | 17 | −0.2 | 0.555 | Heat |
| Brown 7 | | resistance | | | | | | | | resistance |
| Hailan | 217 | Heat | 4.32 | 2678 | 4.1 | 25 | 47 | 0.3 | 1.526 | Heat |
| Brown 8 | | resistance | | | | | | | | resistance |
| Hailan | 244 | Heat | 2.88 | 2772 | 1.57 | 19 | −3 | −2.9 | 0.481 | Heat |
| Brown 9 | | resistance | | | | | | | | resistance |
| Hailan | 363 | Heat | 2.73 | 2974 | 2.67 | 27.06 | 73 | −0.3 | 1.037 | Heat |
| Brown | | resistance | | | | | | | | resistance |
| 10 | | | | | | | | | | |

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A device for judging heat-resistance chickens, comprising: a heat resistance discrimination module and a display module, wherein a discriminant function is stored in the heat resistance discrimination module: y=−1.20×10⁻¹×x₁+7.80×10⁻¹×x₂−6.37×10⁻²×x₃+5.86×10⁻⁴×x₄+9.43×10⁻³×x₅−2.47×10⁻¹×x₆−1.50;
wherein the heat resistance discrimination module is configured to discriminate the heat resistance of chickens according to the blood biochemical index and blood gas index data of chickens before and after mild heat stress treatment;
wherein y represents the heat tolerance of the chicken, x₁ is a first TCHO concentration of the chicken before mild heat stress, x₂ is a second TCHO concentration of the chicken after mild heat stress, x₃ is a Hct level of the chicken after mild heat stress, x₄ is a concentration of CK after mild heat stress in the chicken, x₅ is a difference in the concentration of AST before and after the mild heat stress in the chicken, x₆ is a difference in the concentration of ALB before and after the mild heat stress in the chicken, when an output of y is less than 0, the display module shows that the chicken is heat-labile, and when the output of y is greater than or equal to 0, the display module shows that the chicken is heat-resistant;
wherein the mild heat stress treatment is to transfer the non-heat-stressed chickens to an environment at 32±1° C. and a relative humidity of 60% to 70% for 6 hours.

2. A method for judging heat-resistance chickens, comprising steps of:
Step (1): collecting blood of chickens which are not subjected to heat stress to detect concentrations of TCHO, AST and ALB;
Step (2); treating the chickens in step (1) with mild heat stress: transfering the chickens which are not subjected to heat stress to an environment at 32±1° C. and a relative humidity of 60% to 70%, and keeping for 6 h;
Step (3): collecting the blood of the chickens treated in step (2) to obtain TCHO concentration, Hct level, CK concentration, AST concentration, and ALB concentration; and
Step (4):obtaining based on the parameters of step (1) and step (2): x₁ is the TCHO concentration of the chicken before mild heat stress, x₂ is the TCHO concentration of the chicken after mild heat stress, x₃ is the Hct of the chicken after mild heat stress level, x₄ is the CK concentration after mild heat stress in chickens, x₅ is the difference in AST concentration before and after mild heat stress in chickens, x₆ is the difference in ALB concentration before and after mild heat stress in chickens, and bring the above parameterst into a discriminant function: y=−1.20×10⁻¹×x₁+7.80×10⁻¹×x₂−6.37×10⁻²×x₃+5.86×10⁻⁴×x₄+9.43×10⁻³×x₅−2.47×10⁻¹×x₆−1.50; wherein y represents the heat resistance of the chicken, when the y output is less than 0, the chicken is not heat resistant, when the y output is greater than or equal to 0, the chicken is heat resistant.
